(19) United States
(12) Patent Application Publication
(10) Pub. No.: US 2020/0312465 A1
(43) Pub. Date: Oct. 1, 2020

(54) SYSTEM ARCHITECTURE AND METHODS OF INTELLIGENT MATCHING

(71) Applicant: Purdue Research Foundation, West Lafayette, IN (US)

(72) Inventors: Mohammad S. Rahman, West Lafayette, IN (US); Jitesh H. Panchal, West Lafayette, IN (US); Adam Dachowicz, West Lafayette, IN (US); Siva Chaitanya Chaduvula, West Lafayette, IN (US); Mikhail J. Atallah, West Lafayette, IN (US)

(73) Assignee: Purdue Research Foundation, West Lafayette, IN (US)

(21) Appl. No.: 16/829,746

(22) Filed: Mar. 25, 2020

Related U.S. Application Data
(60) Provisional application No. 62/823,179, filed on Mar. 25, 2019.

(51) Int. Cl. G16H 80/00; G16H 40/00; G06F 16/906

(52) U.S. Cl. CPC G16H 80/00; G06F 16/906; G16H 40/00

(57) ABSTRACT

A non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method additionally includes classifying, using cluster algorithms, the service consumers from the first data into a plurality of groups. Further, the method includes calculating a compatibility success score between each service provider and each group of the plurality of groups. Moreover, the method includes calculating a historical arrival rate for the each group of the plurality of groups. The method also includes assigning a chosen service provider to a prospective service consumer based on a determined compatibility success score of the chosen service provider.

### CROSS-REFERENCE TO RELATED APPLICATIONS

The present U.S. patent application is related to and claims the priority benefit of U.S. Provisional Patent Application Ser. No. 62/823,179, filed Mar. 25, 2019, the contents of which is hereby incorporated by reference in its entirety into this disclosure.

### GOVERNMENT FUNDING

This invention was made with government support under IIP1744195 awarded by National Science Foundation. The government has certain rights in the invention.

### BACKGROUND

This section introduces aspects that may help facilitate a better understanding of the disclosure. Accordingly, these statements are to be read in this light and are not to be understood as admissions about what is or is not prior art.

Recent trends in healthcare payment models make this a particularly attractive time to introduce such an algorithm to the market. For reference, consider the Center for Medicare and Medicaid Services (CMS), the public payer for both Medicare and Medicaid. CMS is the largest payer of healthcare expenditure in the U.S. CMS has introduced new value-based payment models such as bundled payments for different types of surgeries, including orthopedic and cardiac surgeries. Bundled payments, or episodic-care payments, are single payments based on a set price derived from historical data. If the cost of the episodic care is less than the set price, health facilities can keep the difference. However, if the cost of care is more than the set price (due to complications, readmissions, or other factors), health facilities lose the difference. The inherent financial risk in this payment model motivates healthcare facilities to improve care quality. Traditional insurance companies such as Blue Cross Blue Shield and United Healthcare are using instruments such as price transparency tools to provide more information to their members, including patient-reported scores and physician ratings. This additional information enables patients to shop for medical services and helps in making better healthcare decisions. Employers offering healthcare to their employees are shifting to self-insured solutions, in turn eliminating the involvement of insurance companies in the provision of healthcare coverage and opting instead for direct contracting with health facilities.

Existing scheduling tools are predominantly patient-facing. For instance, Zocdoc and DocASAP provide lists of available physicians based on user input. However, these tools do not offer matching as a service. Private insurance companies such as Blue-Cross Blue Shield and United As discussed above, healthcare insurance companies have introduced a patient-facing scheduling service, price transparency tools, to assist policy members when shopping for medical services. These price transparency tools are also offered by non-insurance companies such as CastLight and Vitals. However, these tools are mainly geared towards self-insured employers, and they evaluate physicians using patient reported outcomes and medical claims data. New players such as Amino are introducing claims data-driven suggestions based on patient volume, gender and age. This solution is flawed as a high-volume physician may not necessarily provide the best outcomes at the best price. None of these patient-facing solutions are based on clinical data, and hence they do not help patients make informed healthcare decisions. Consequently, these solutions are experiencing low acceptance among patients and do not solve the technical challenges addressed by the algorithm presented.

Hospital-reported data has been used to a limited extent by some companies. Clinical data derived from patients is recorded and closely guarded by health facilities. Kyruus is a provider-facing tool that consolidates physician data at an enterprise level. This tool also collects self-reported physician specialties and uses this information to schedule patients with physicians. This ontology-based scheduling does not consider patient medical history, comorbidities, or other relevant features. Hence, this approach is not personalized to the patient, and more importantly not aimed at reducing outcome variations. Hence, the existing solutions do not succeed at scheduling patients with physicians in a way that reduces healthcare costs while improving quality of care and overall patient outcomes.

Prior art in computer science, within the sub-field of algorithms for intelligent matching in artificial intelligence, dealt with architectures of assigning workers to tasks in a framework where the quality of the assignment of each worker to each task is either already known or easily derivable, in a numerically quantified matrix form that is available for use by the intelligent algorithm. However, none of the conventional solutions in the field of artificial intelligence address scenarios where a quality of a worker in carrying a specific task is neither known nor computable.

### SUMMARY

Various embodiments of the present application relate to software architectures that address the quantification of how good a worker x is in carrying out a task y, when neither is known, nor computable (e.g., because there is no past track record for x); but must instead be learned over time as x carries out various different tasks. Various embodiments of the present application (i) handle the assignment of tasks to workers as workers evolve over time from being initially unknown to becoming better known and eventually becoming well-known; (ii) carry out learning and updating, over time, of how good each worker is at each task, and uses such knowledge to make future assignments; (iii) discover changes (improvement or worsening) of each worker's skill at carrying out each task (so our scheme does not assume that a worker has an unchanging skill towards a task, rather, it tracks such skills as they change over time in either direction).

One aspect of the present application relates to a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method additionally includes classifying, using cluster algorithms, the service consumers from the first data into a plurality of groups. Further, the method includes calculating a compatibility success score between each service provider and each group of the plurality of groups. Moreover, the method includes calculating a historical arrival rate for the each group of the plurality of groups. The method also includes assigning a chosen service provider to a prospective service consumer based on a determined compatibility success score of the chosen service provider, wherein the determined compatibility success score is the compatibility success score between the chosen service provider and a selected group of the plurality of group, wherein the selected group includes the characteristics of the prospective service consumer. In some embodiments, using cluster algorithms includes using approaches based on techniques such as machine-learning algorithms, statistics-based techniques, etc.

Another aspect of the present application relates to a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method additionally includes classifying, using cluster algorithms, the service consumers from the first data into a plurality of groups. The method further includes calculating a compatibility success score between each service provider and each group of the plurality of groups, wherein the calculating includes obtaining statistical properties of distribution of a performance indicator of the at least one service provider associated with an entrusted group, wherein the at least one service provider has previously provided service to the entrusted group. The calculating also includes ranking the at least one service providers based on the obtained statistical properties of distribution of the performance indicator. Next, the method includes calculating a historical arrival rate for the each group of the plurality of groups. Moreover, the method includes assigning a chosen service provider to a prospective service consumer based on a determined compatibility success score of the chosen service provider, wherein the determined compatibility success score is the compatibility success score between the chosen service provider and a selected group of the plurality of group, wherein the selected group includes the characteristics of the prospective service consumer. In some embodiments, using cluster algorithms includes using approaches based on techniques such as machine-learning algorithms, statistics-based techniques, etc.

Still another aspect of the present application relates to a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method additionally includes classifying, using cluster algorithms, the service consumers from the first data into a plurality of groups. The method further includes calculating a compatibility success score between each service provider and each group of the plurality of groups, wherein the calculating includes obtaining statistical properties of distribution of a performance indicator of the at least one service provider associated with an entrusted group, wherein the at least one service provider has previously provided service to the entrusted group. The calculating also includes ranking the at least one service providers based on the obtained statistical properties of distribution of the performance indicator. Next, the method includes calculating a historical arrival rate for the each group of the plurality of groups. Moreover, the method includes assigning a chosen service provider to a prospective service consumer based on a determined compatibility success score of the chosen service provider, wherein the determined compatibility success score is the compatibility success score between the chosen service provider and a selected group of the plurality of group, wherein the selected group includes the characteristics of the prospective service consumer. The assigning includes calculating a probability of the prospective service consumer arriving at a service time window based on the historical arrival rate of the selected group. Next, the assigning includes calculating a reward for a reinforcement algorithm based on a selection criteria, wherein the selection criteria includes the compatibility success score. Moreover, the assigning includes imposing a penalty function to the reinforcement algorithm for using the chosen service provider, wherein the penalty function is calculated based on unavailability of the chosen service provider. In some embodiments, using cluster algorithms includes using approaches based on techniques such as machine-learning algorithms, statistics-based techniques, etc.

### BRIEF DESCRIPTION OF THE DRAWINGS

One or more embodiments are illustrated by way of example, and not by limitation, in the figures of the accompanying drawings, wherein elements having the same reference numeral designations represent like elements throughout. It is emphasized that, in accordance with standard practice in the industry, various features may not be drawn to scale and are used for illustration purposes only. In fact, the dimensions of the various features in the drawings may be arbitrarily increased or reduced for clarity of discussion.

FIG. 1 illustrates a workflow for the system architecture.

FIG. 2 illustrates a performance based classifier where “best” indicates above-average performance in price and quality, and “worst” indicates below-average performance in price and quality.

FIG. 3 illustrates potential patient subgroups using patient-specific features.

FIG. 4 illustrates block diagrams for systems 110 and 120.

FIG. 5 illustrates block diagrams for system 140.

FIG. 6 illustrates an administrator dashboard.

FIG. 7 illustrates a second administrator dashboard.

FIG. 8 illustrates a physician dashboard.

FIG. 9 illustrates a block diagram for system 150.

FIG. 10 illustrates a physician n having different scores in different patient groups for a specific elective medical procedure.

FIG. 11 illustrates flow chart for patient-physician matching.

FIG. 12 illustrates flowchart for caregap identification.

FIG. 13 illustrates flowchart for recommendation of care teams.

FIG. 14 illustrates one example of a computing or processing node 1500 for operating a method or a software architecture in accordance with the present application.

### DETAILED DESCRIPTION

The following disclosure provides many different embodiments, or examples, for implementing different features of the present application. Specific examples of components and arrangements are described below to simplify the present disclosure. These are examples and are not intended to be limiting. The making and using of illustrative embodiments are discussed in detail below. It should be appreciated, however, that the disclosure provides many applicable concepts that can be embodied in a wide variety of specific contexts. In at least some embodiments, one or more embodiment(s) detailed herein and/or variations thereof are combinable with one or more embodiment(s) herein and/or variations thereof.

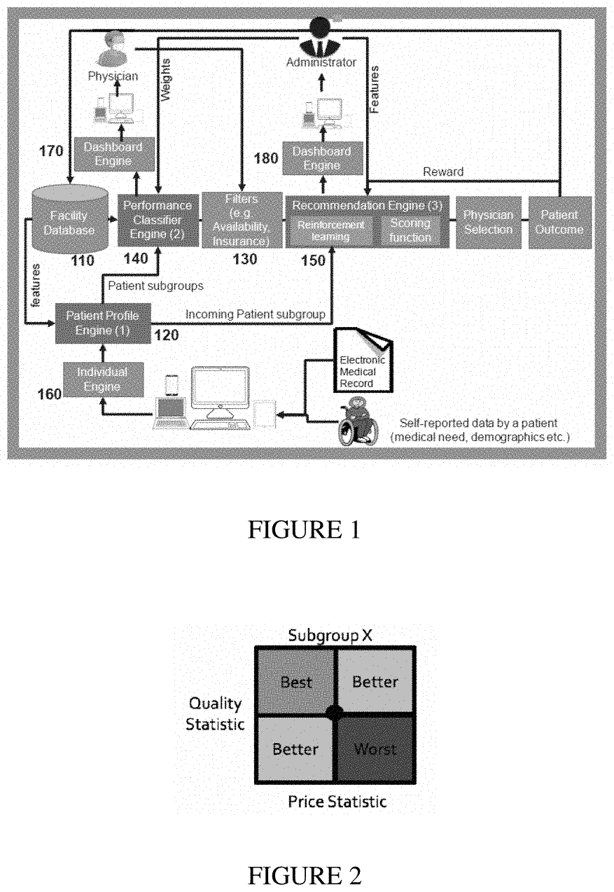

FIG. 1 illustrates the entire workflow 100 of the proposed patient-physician matching. This workflow mainly consists of three engines: the Profile engine 120, the Classifier engine 140, and the Recommendation engine 150. The data required to run these engines is derived from the facility database 110, which is connected to various data warehouses that exist within a health facility or system. This workflow could be supported by different computing environments and can have multiple interfaces through which data is collected or through which processed information is retrieved.

Systems 110 and 120 are presented in FIG. 4. System 110, the facility database, is connected to various data sources including claims 111, physician data 112, patient-reported outcomes 113, and clinical outcomes 114. In addition to these sources, this database is also connected to data sources containing patient demographic data 115 such as gender, age, zip code, and social determinants 116 such as access to care, income status, quality of life, and medical history of the patient. This rich historical data on the patient population is used to identify the outcome-influencing patient features. Such features are used to classify patients into different subgroups. For instance, the outcome of patients needing a knee replacement is heavily influenced by comorbidities such as obesity and hypertension, along with demographic features such as gender and age. These sub-groups are used in the classification and recommendation engines to provide appropriate patient-physician matching.

For simplicity, the data structure for System 110 is assumed to be a database. However, System 110 could take other forms, such as flat file, data stored in memory, etc.

In some embodiments, System 110 could be a Relational Database Management System (RDBMS) or refer to the OMOP Common data model. In RDBMS, the different data sources are linked through a reference column and thereby helps in collecting the data from multiple data sources in a few operations.

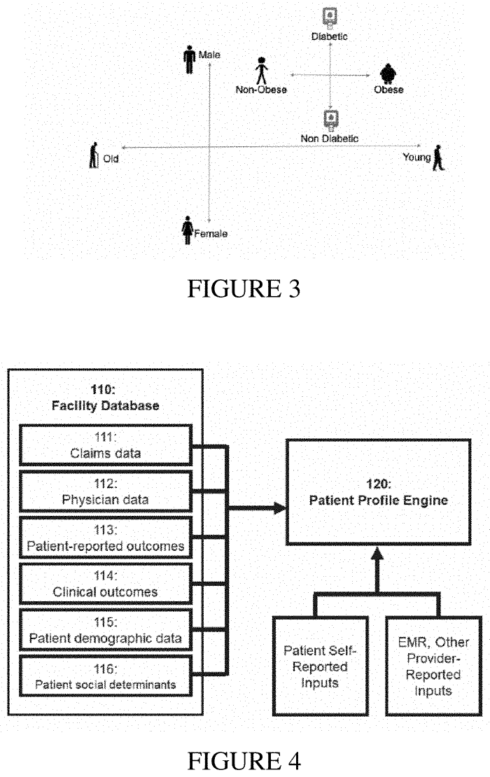

Classification of Patient Population: System 120, the patient profile engine, processes the data stored in System 110 and determines the outcome-influencing features for different elective medical procedures. These features could be related to patient demographics, social determinants, or other features stored in the facility database and can be derived from relevant data using machine learning algorithms. Based on the selected features, the patient population is classified into different subgroups. A graphical representation of this patient subgrouping is illustrated in FIG. 3.

An incoming patient makes a request for a physician using an electronic device such as laptop computer, desktop computer, notebook, or mobile phone, and this information is stored in an individual engine, System 160. In making such a request, the patient may enter their personal data, including demographics, social determinants, and physician preferences. In addition to this, the patient uploads their electronic medical record from which the clinical-related data, such as medical conditions and medical history, is derived. This clinical data could also be self-reported. The individual engine 160 processes the patient-provided data into a request to the Patient Profile Engine, System 120.

Alternatively, the patient can provide patient-related data via phone to the physician's assistant or receptionist of a health facility, or to a physician who enters it into the System 160.

The individual engine 160 processes the data from the incoming patient and sends it to the Patient Profile Engine 120. With this information, System 120 determines the patient subgroup of the incoming patient and shares it with recommendation engine 150.

Classification of Physician Performance: The Performance Classifier Engine 140 analyzes both clinical and non-clinical data on the previously offered care procedures for all physicians. It considers outcome- and outcome-influencing characteristics, including:

1. Clinical outcomes, such as inpatient length-of-stay, discharge disposition, and/or infection rates,

2. Patient-reported outcomes, such as ease of recovery, pain, and impression of physician quality of care,

3. Productivity metrics, such as physician case volume or profit generated for the health facility, and

4. Utilization metrics, measures of health facility resource use per case/per physician.

In addition to this, it receives information on patient subgroups from the Patient Profile engine 120.

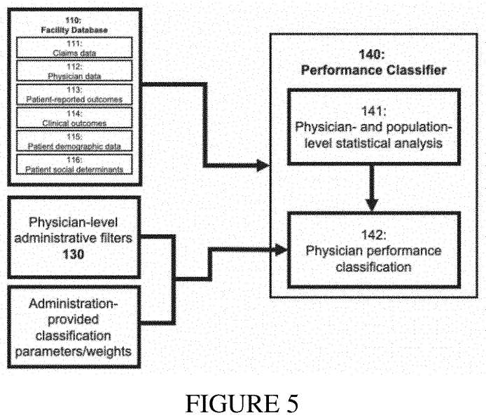

System 140 (see FIG. 5) is composed of a physician- and population-level statistical analysis subroutine 141, as well as a physician performance classification subroutine, 142. The system takes in historical data on the patients treated by the physicians stored in System 110.

Physician- and population-level statistical analysis, 141, provides a summary statistical analysis over the past patients treated by each physician. The features considered for this physician-level analysis could be a subset of the features mentioned in [0033]. A healthcare administrator provides a list of features and associated time period for this analysis.

Physician- and population-level statistical analysis, 141, also provides summary statistical analyses over the incoming patients by each patient subgroup. The features for this population-level analysis could be a subset of the features mentioned in [0029]. A healthcare administrator provides their list of features and associated time period for this analysis.

Physician performance classification 142, takes physician-level and population-level summary statistics along with a feature for segmenting physicians into different classes. This segmenting feature could be a single feature or a combination of features. An illustration of segmenting physicians into classes (“worse,” “better,” and “best,”) is illustrated in FIG. 2. This segmentation could be performed through comparison of a physician's past performance in one or more quality/cost metrics to that of a patient population.

In an embodiment, all statistical analysis performed in Physician performance classification 142 could be performed on the raw data or normalized data. Also, the analysis is performed only when the number of data points for each physician are greater than or equal to a threshold set to insure statistical confidence or to prevent re-identification of data. If there are fewer data points than the threshold allows, the physician is classified as a separated class, “new.”

Since this analysis returns distributions over features for each physician, the classification of a physician into different classes could be performed using A/B hypothesis testing, or through computation of distribution distance given by methods such as Kullback-Leibler divergence.

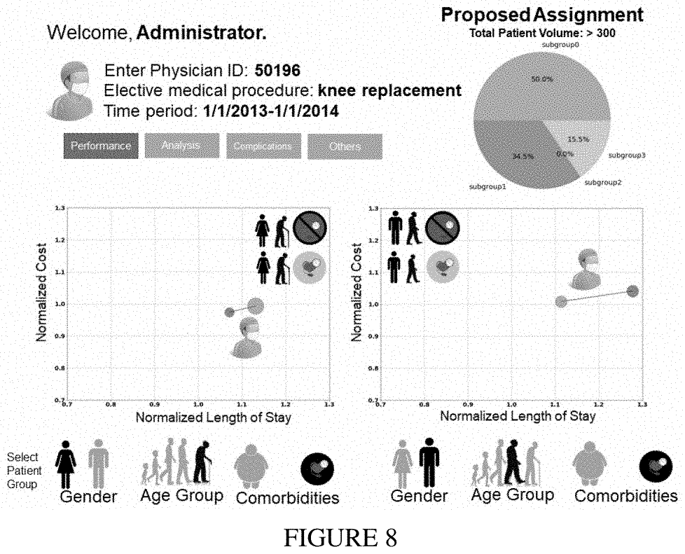

The Performance Classifier Engine provides a feedback report on each individual physician to the dashboard engine 170. To illustrate, a version of the feedback is shown in FIG. 8. This feedback could include physician performance over different time periods and/or benchmarking of physicians.

A physician provides appropriate credentials to the dashboard engine 170 to access their feedback reports. The access credentials could be in the form of username/password or could have multi-factor authentication. In addition to this feedback report, physicians also receive information on training programs that could help improve their care services.

A physician, or physician's assistant on behalf of a physician, enters their preferences to System 130 using an electronic device such as laptop computer, desktop computer, notebook, or mobile. This may contain data including:

1. Accepted insurance plans,

2. Time availability,

3. Privileges, and

4. Flexibility in the patient load when compared with the previous time period.

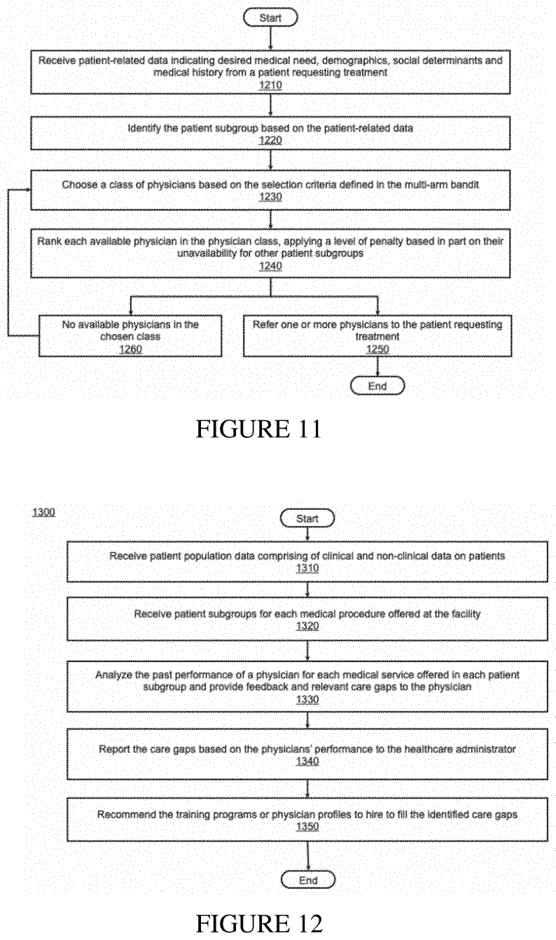

Physician-patient matching: FIG. 11 is a flow chart illustrating a process for matching physicians to patients. The process may include different or additional steps than those described in conjunction with FIG. 11.

In the embodiment illustrated in FIG. 11, the individual engine 160 receives patient-related data (as listed in [0033]) from the patient requesting treatment 1210. Using this data, Patient Profile Engine 120 determines the patient subgroup 1220.

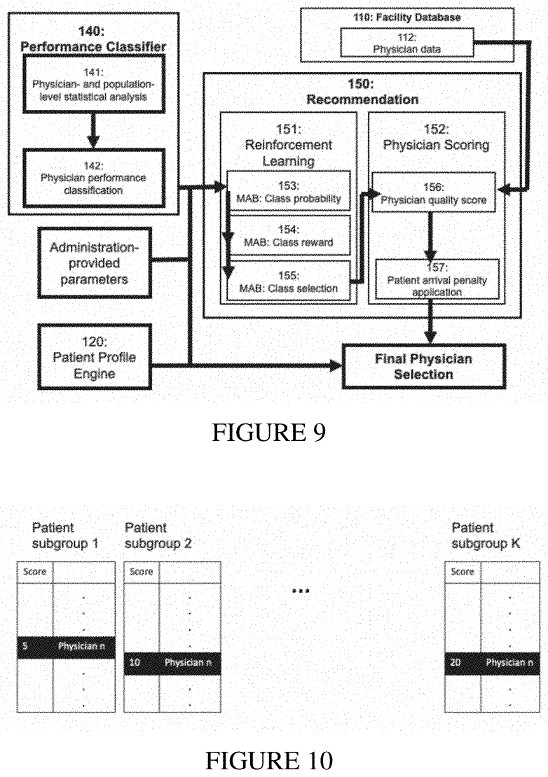

The recommendation engine system 150 uses the patient subgroup to recommend best-suited physicians to the patient (see FIG. 9). It comprises of two sub-systems: reinforcement learning 151 and the scoring function 152.

The recommendation engine drops the unavailable physicians for the time period requested by the incoming patient. Further, it applies the filters provided by the physicians or administrators on patient selection.

Sub-system 151 employs a reinforcement-based learning algorithm to select the most appropriate physician class to maximize the health facility's improvement of a particular feature, while exploring the physician classes to gain more information. The feature to improve could be quality, cost, or a combination of both, and it is provided by the healthcare administrator. The reinforcement-based learning algorithm implemented in the present embodiment is a Lower Confidence Bound (LCB) Multi-Arm Bandit algorithm. LCB is deterministic in nature and requires the feedback/reward to be immediate. In the current algorithm, we thus use physician selection by a patient. More details on LCB Multi-arm bandit algorithm are provided in Table 5 below.

In some embodiments, we may need to choose feedback/reward that is not instantaneous. For example, patient outcome could be used as a reward for choosing a physician class. In such scenarios, we may use techniques such as Thompson sampling to handle this form of delayed feedback.

Sub-system 152 scores the physicians belonging to the class selected by 151 using a scoring function. This scoring function could take as input a combination of different out-come features and uncertainty in the patient arrivals. The scoring function could further be defined over relevant summary statistics of these features, such as mean, median, variance, or higher moments. In one embodiment, this function may be a weighted average of the medians for each quality metric after normalization, where the weights are assigned by the healthcare administrator. In another embodiment, this function may take medical outcome or resource utilization variance, total patient case load, or other performance descriptors as inputs. In the preferred embodiment, the subroutine outputs quality digests for each physician, for each patient subgroup, as scalars. However, the scoring functions may be output as vectors or arrays if multiple dimensions are necessary for the final recommendation.

Sub-system 152 also accommodates the uncertainty in the arrival of patient population. In the embodiment, a physician's score is modified from the original physician quality digest for the incoming patient subgroup by:

1. computing penalty terms reflecting the difference between a physician's quality digest for an incoming patient subgroup and the “next best” physician for that subgroup as determined by the ordered list of quality digests,

2. multiplying each of these penalty terms by the probability of a patient from that sub-group arriving during the time interval that the physician would be occupied by the current patient if assigned, and

3. subtracting the sum of these terms from the physician's original score.

These terms may be modified further by administrator-defined weights for each sub-group. In the preferred embodiment, the list of physicians ordered by these scores provides the final recommendation output by the algorithm. An illustration of this is shown in FIG. 10.

In case there are no available physicians in the chosen physician class, then the multi arm bandit chooses the next best alternative of physician class. This is continued until the “best”, “better” and “new” physician classes are exhaustively searched. If there are no available physicians from these classes, in one embodiment, the patient is intimated on the unavailability of physicians for the chosen time period. In another embodiment, the system chooses from “worse” physician class and recommends to the patient.

The incoming patient selects a physician from the recommendations provided by System 150 and confirms the appointment. After confirmation, the patient and physician receive a notification on appointment details (date, time, and place of visit.)

As per the appointment, the patient receives the desired treatment from the physician. The outcomes corresponding to this treatment are fed back to the recommendation engine 150 as well as the facility database 110.

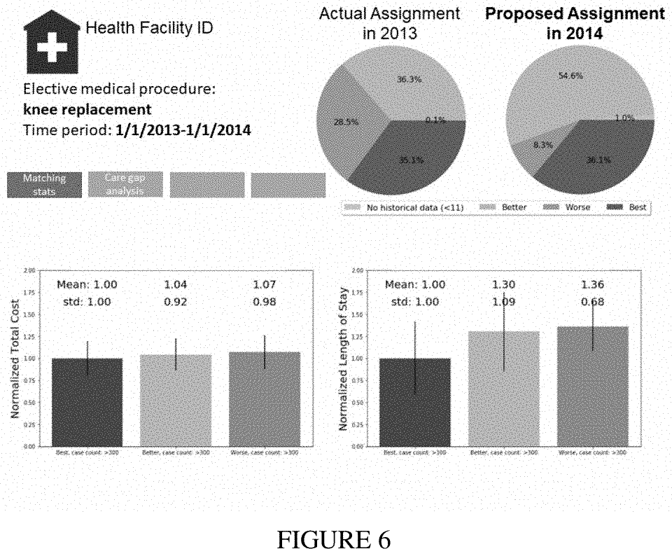

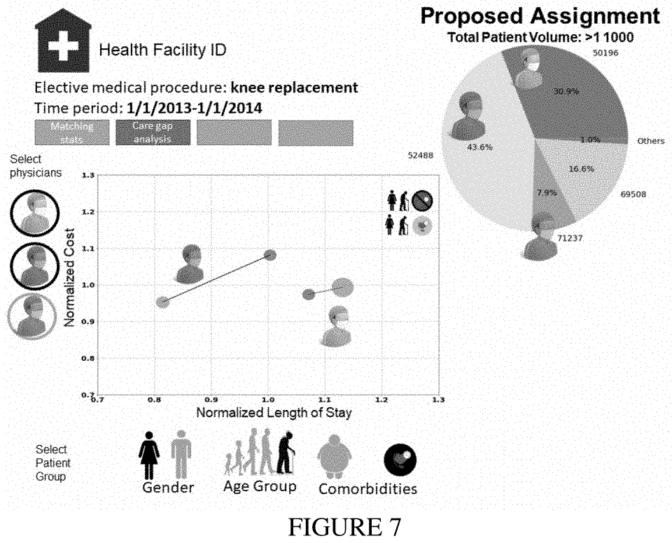

The recommendation engine 150 also provides a facility-wide report to the dashboard engine. This report comprises of details as illustrated in FIG. 6 and FIG. 7.

An administrator provides appropriate credentials to the dashboard engine 170 to access the report. The access credentials could be in the form of username, password or can have multi-factor authentication.

Care gap identification: FIG. 12 is a flow chart illustrating a process for identifying the care gaps associated with different medical procedures. The process may include different or additional steps than those described in conjunction with FIG. 12.

System 110 provides both clinical and non-clinical data on the patient population 1310. System 120 provides the patient sub-groups associated with the patient population for each medical procedure offered at the facility 1320. System 140 uses these patient subgroups and analyses the past performance of a physician for each medical service offered in each patient subgroup 1330. At a facility level, this analysis is reported to healthcare administrator along with any care gaps 1340. Training programs for physicians or desired physician profiles for hiring are recommended if care gaps are identified 1350.

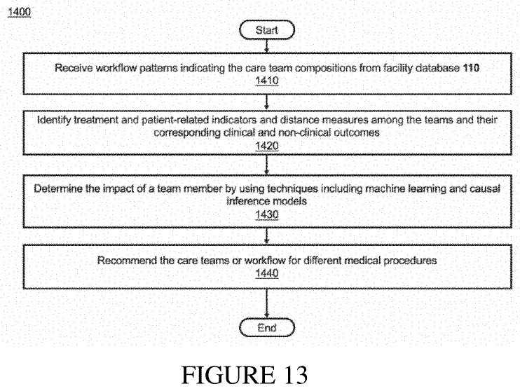

Team-based care identification: FIG. 13 is a flow chart illustrating a process for determining the impact of a team member. The process may include different or additional steps than those described in conjunction with FIG. 13.

System 110 provides both clinical and non-clinical data on the workflow patterns including team composition 1410. Treatment and patient-related indicators that affect clinical outcomes, along with distance measures between teams, are identified using unsupervised learning or other appropriate techniques 1420. The impact of a team member is defined using techniques from causal inference models 1430. Based on these relationships, care teams that offer higher quality are recommended to the healthcare facilities 1440.

In addition to team-based care analysis, this method can be used to analyze the impact of a procedural change such as new equipment, training procedures in the care delivery on care quality. Such a method can be further extended for different medical procedures.

One of ordinary skilled in the art would recognize that the methodology described in the above example is programmed into a software architecture which is differentiated by various protocols, wherein each discretized protocol is configured to execute a different method. A multitude of tables exemplify the input and outputs associated with each applicable protocol, in accordance with one or more embodiments.

TABLE 1
Performance Classifier Engine

| | |
|---|---|
| Relevant Classes: | subgroup_analysis(), Rating() |
| Input: | physician list, can episode-level historical quality |
| | metrics for each relevant patient, subgroup, for |
| | each physician in the physician list, list of all patient |
| | subgroups in the population, parameters for segmenting |
| | physicians into classes by comparing physician |
| | performance for each quality metric to the population |
| | of physicians in the physician list. |
| Output: | physician-specific statistical analysis for each |
| | patient subgroup. |
| | physician-specific classification for each patient |
| | subgroup. |
| Sub-routines: | Physician and population-level statistical analysis |
| | Physician performance classification |

TABLE 2
141: Physician- and population-level statistic analysis

| | |
|---|---|
| Input: | physician list, patient subgroup list, historial quality metrics |
| | for each physician. |
| Output: | physician population summary statistics for each quality |
| | metric, physician summary statistics for each quality metric |
| 1.1 | For each patient subgroup, for each physician, compute |
| | summary statistics for each historical quality metric in the |
| | dataset. Call these statistics physician-level statistics. |
| 1.2 | For all eases for each patient subgroup recorded in the dataset, |
| | compute summary statistics for each historical quality metric |
| | in the dataset. Call these statistics population-level statistics. |
| 1.3 | Return, as array, physician-level statistics, for each patient |
| | subgroup, for each physicians. |
| 1.4 | Return, as array, population-level statistics, for each patient |
| | subgroup. |

TABLE 3
142: Physician performance classification

| | |
|---|---|
| Input: | physician population summary statistics for each quality |
| | metric, physician summary statistics for each quality |
| | metric, physician classification, parameters. |
| Output: | classy assigned to each physician, for each patient subgroup. |
| 1.1 | Define potential physician classifications based on user input |
| | parameters. |
| 1.2 | For each patient subgroup, for each physician, compute the |
| | classification of the physician using the input parameters and |
| | the summary statistics of the physician and the population. |
| 1.3 | Return, as array, the classification of each input physician, |
| | for each patient subgroup. |

TABLE 4
153 Multi-Arm Bandit: Determine probability on
the given physician class

| | |
|---|---|
| Input: | Rank dataframe (R), incoming patient subgroup, physician |
| | class |
| Output: | Determine probability of each physician class |
| 1.1 | Filter the Rank dataframe using incoming patient subgroup |
| | and unavailable physicians |
| 1.2 | Determine the ratio of physicians in the given physician |
| | class to the total number of physicians in the filtered rank |
| | dataframe |

TABLE 5
154 Multi-Arm Bandit: Reward

| | |
|---|---|
| Input: | Historical patient population data, actual patient data, cost |
| | feature, quality feature |
| Output: | reward |
| 1.1 | Determine the mean values of historical patient population |
| | using the cost and quality features provided by the healthcare |
| | administrator |
| 1.2 | Determine the values of corresponding to the cost and quality |
| | feature of the actual patient |
| 1.3 | If the cost and quality are better then the physician receives |
| | highest reward. If only the cost or quality are better then the |
| | physician receives a reward. Else no reward for the physician. |

TABLE 6
155 Multi-Arm Bandit: UCB1

| | |
|---|---|
| Input: | Rank dataframe, cost feature, quality feature, patient subgroup, |
| | actual patient data |
| Output: | best alternative class of physicians |
| 1.1 | Use the feature provided by the healthcare administrator to |
| | classify physicians into the four classes: Best, Better, New and |
| | Worse. |
| 1.2 | Remove the undesired physician class (“worse”) from the |
| | Rank dataframe |
| 1.3 | Choose the best alternative of physician class using the |
| | probabilities calculated in table 4 and suggest the best |
| | physicians from that class |
| 1.4 | Record the reward using the algorithm listed in Table 5 and |
| | number of times the physician class (n_{pc}) was chosen |
| 1.5 | Choose the physician class that minimizes |
| | $\frac{sum of all rewards}{{n}_{pc}} - \sqrt{\frac{2 logi}{{n}_{pc}}}$ |

TABLE 7
152: Scoring Function Sub-engine

| | |
|---|---|
| Relevant Classes: | Rating() |
| Input: | candidate physician list (as output by the |
| | Reinforcement Learning Sub-Engine), physician- |
| | specific subgroup achievement statistics and |
| | classifications (as output by the Performance |
| | Classifier Engine) physician availability at |
| | run-time, physician expected case load per time |
| | period, incoming patient subgroup, list of all |
| | patient subgroups in the population, relative |
| | weighting of each quality metric for final scoring |
| | output, relative weighting for random arrival-based |
| | penalty for final scoring output. |
| Output: | ordered list of physician recommendation scores |
| | for the incoming patient. |
| Sub-routines: | Physician quality scoring |
| | Application of patient-arrival-estimation penalties |
| | to physician scores and patient recommendation |

TABLE 8
156: Physician quality scoring

| | |
|---|---|
| Input: | physician list, patient subgroup list, physician summary |
| | statistics for each quality metric, weighting for each |
| | physician summary statistic response, physician population |
| | summary statistics for each quality metric. |
| Output: | Quality digest for each patient subgroup, for each physician, |
| | for each procedure considered. |
| 1.1 | For each patient subgroup, for each physician, define the |
| | relevant summary statistics for computing physician quality |
| | scores. |
| 1.2 | Normalize relevant statistics with respect to the population- |
| | level summary statistics. |
| 1.3 | Weight relevant statistics according to input weighting. |
| 1.4 | For each patient subgroup, for each physician, compute a |
| | quality digest as a function of the weighted, normalized |
| | relevant statistics. For instance, the digest may be computed |
| | as the weighted average of the relevant statistics. |
| 1.5 | Return, as array, the quality digests for each physician, for |
| | each patient subgroup. |

TABLE 9
157: Application of patient arrival-estimation penalties to
physician scores and patient recommendation

| | |
|---|---|
| Input: | physician list, incoming patient, recommended physician |
| | classification for the incoming patient, ordered physician |
| | quality digests, physician availability at run-time, physician |
| | capacity over time period specified, arrival penalty function |
| | parameters. |
| Output: | Physician list and relative scores serving as the final |
| | recommendation output for the incoming patient. |
| 1.1 | Query, from ordered physician quality digests, the quality |
| | digests for each physician, for the incoming patient |
| | subgroup. |
| 1.2 | For each physician in the physician list.: |
| | 1.2.1 compute expected work time lost to treat incoming |
| | patient. |
| | 1.2.2 for each patient subgroup: |
| | 1.2.2.1 identify physician with next-highest quality |
| | digest for the patient subgroup. |
| | 1.2.2.2 compute some user-calibrated distance between the |
| | physician's and next-highest physician's digests. |
| | 1.2.2.3 weight this distance with some representation of the |
| | likelihood a patient of this subgroup will arrive during the |
| | time taken to treat the incoming patient. |
| | 1.2.3 Take some linear combination of the computed |
| | weighted distances for each subgroup, and apply the |
| | sum as a penalty to the physician's quality digest for the |
| | incoming patient subgroup. Call this result the physician's |
| | recommendation score. |
| 1.3 | Return, as an ordered list, the physicians ranked according |
| | to the recommendation scores computed in step 1.2. |

### Example 1

One example of the present application relates to a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. Additionally, the method includes classifying, using cluster algorithms, the service consumers from the first data into a plurality of groups. Moreover, the method includes calculating a compatibility success score between each service provider and each group of the plurality of groups. Further, the method includes calculating a historical arrival rate for the each group of the plurality of groups. The method also includes assigning a chosen service provider to a prospective service consumer based on a determined compatibility success score of the chosen service provider, wherein the determined compatibility success score is the compatibility success score between the chosen service provider and a selected group of the plurality of group, wherein the selected group comprises the characteristics of the prospective service consumer.

In one or more embodiments, the receiving the first data further includes receiving a second data, wherein the second data includes: at least one of characteristics of service providers or preferences of service consumers. In one or more embodiments, the classifying, using the cluster algorithms, the service consumers from the first data into the plurality of groups includes classifying, using the cluster algorithms, the service consumers from the first data and the second data into the plurality of groups. In some embodiments, using cluster algorithms includes using approaches based on techniques such as machine-learning algorithms, statistics-based techniques, etc.

In one or more embodiments, the calculating the compatibility success score between the each service provider and the each group of the plurality of groups includes obtaining statistical properties of distribution of a performance indicator of the at least one service provider associated with an entrusted group, wherein the at least one service provider has previously provided service to the entrusted group. The calculating further includes ranking the at least one service providers based on the obtained statistical properties of distribution of the performance indicator.

In at least one embodiment, the calculating the compatibility success score between the each service provider and the each group of the plurality of groups includes assigning statistical values to the under-represented service provider based on standard missing data procedures, where an under-represented service provider of the at least one service provider does not have adequate statistical representation of the performance indicator in the entrusted group.

In at least one embodiment, the performance indicator includes at least one of service cost, service time, service quality, service satisfaction, or service compliance. In such embodiment, the calculating further includes ranking the at least one service providers based on the obtained statistical properties of distribution of the performance indicator, wherein at least one of the performance indicators are weighted by a user.

In the embodiment where the performance indicator includes at least one of service cost, service time, service quality, service satisfaction, or service compliance, the assigning the chosen service provider to the prospective service consumer based on the determined compatibility success score of the chosen service provider includes calculating a probability of the prospective service consumer arriving at a service time window based on the historical arrival rate of the selected group. The determined compatibility success score is the compatibility success score between the chosen service provider and the selected group of the plurality of group. The selected group comprises the characteristics of the prospective service consumer. The assigning further includes calculating a reward for a reinforcement algorithm based on a selection criteria, wherein the selection criteria includes the compatibility success score. Furthermore, the assigning includes imposing a penalty function to the reinforcement algorithm for using the chosen service provider, wherein the penalty function is calculated based on unavailability of the chosen service provider.

In at least one embodiment, the reward is a reference parameter. In at least one embodiment, the selection criteria further includes preferences of the service consumers. In at least one embodiment, the calculating the reward for the reinforcement algorithm based on the selection criteria includes calibrating the reward based on external penalty functions from external reinforcement algorithm, wherein the external reinforcement algorithm and the reinforcement algorithm are part of a homogenous system implementation. The selection criteria includes the compatibility success score.

One of ordinary skilled in the art would recognize that the methodology described in the above example is programmed into a software architecture which is differentiated by various protocols, wherein each discretized protocol is configured to execute a different method.

### Example 2

Another example of the present application relates to a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method additionally includes classifying, using cluster algorithms, the service consumers from the first data into a plurality of groups. Additionally, the method includes calculating a compatibility success score between each service provider and each group of the plurality of groups, wherein the calculating includes obtaining statistical properties of distribution of a performance indicator of the at least one service provider associated with an entrusted group, wherein the at least one service provider has previously provided service to the entrusted group. Additionally, the calculating includes ranking the at least one service providers based on the obtained statistical properties of distribution of the performance indicator. Moreover, the calculating includes calculating a historical arrival rate for the each group of the plurality of groups. Further, the calculating includes assigning a chosen service provider to a prospective service consumer based on a determined compatibility success score of the chosen service provider, wherein the determined compatibility success score is the compatibility success score between the chosen service provider and a selected group of the plurality of group, wherein the selected group includes the characteristics of the prospective service consumer. In some embodiments, using cluster algorithms includes using approaches based on techniques such as machine-learning algorithms, statistics-based techniques, etc.

In one or more embodiments, the performance indicator includes at least one of service cost, service time, service quality, service satisfaction, or service compliance. In one or more embodiments, the assigning the chosen service provider to the prospective service consumer based on the determined compatibility success score of the chosen service provider, wherein the determined compatibility success score is the compatibility success score between the chosen service provider and the selected group of the plurality of group, wherein the selected group includes the characteristics of the prospective service consumer, includes: calculating a probability of the prospective service consumer arriving at a service time window based on the historical arrival rate of the selected group. The assigning further includes calculating a reward for a reinforcement algorithm based on a selection criteria, wherein the selection criteria includes the compatibility success score. Moreover, the assigning includes imposing a penalty function to the reinforcement algorithm for using the chosen service provider, wherein the penalty function is calculated based on unavailability of the chosen service provider.

In at least one embodiment, the reward is a reference parameter. In at least one embodiment, the selection criteria further includes preferences of the service consumers.

One of ordinary skilled in the art would recognize that the methodology described in the above example is programmed into a software architecture which is differentiated by various protocols, wherein each discretized protocol is configured to execute a different method.

### Example 3

Yet another example of a non-transitory computer-readable medium encoded with a computer-readable program, which, when executed by a processor, will cause a computer to execute a method of intelligent matching, wherein the method includes receiving a first data from a plurality of service vendors, wherein the first data includes performance metrics of service providers and characteristics of service consumers. The method further includes classifying, using cluster algorithms, the service consumers from the first data into a plurality of groups. Additionally, the method includes calculating a compatibility success score between each service provider and each group of the plurality of groups, wherein the calculating includes obtaining statistical properties of distribution of a performance indicator of the at least one service provider associated with an entrusted group, wherein the at least one service provider has previously provided service to the entrusted group. The calculating additionally includes ranking the at least one service providers based on the obtained statistical properties of distribution of the performance indicator. The method additionally includes calculating a historical arrival rate for the each group of the plurality of groups. In some embodiments, using cluster algorithms includes using approaches based on techniques such as machine-learning algorithms, statistics-based techniques, etc.

Next, the method includes assigning a chosen service provider to a prospective service consumer based on a determined compatibility success score of the chosen service provider, wherein the determined compatibility success score is the compatibility success score between the chosen service provider and a selected group of the plurality of group, wherein the selected group includes the characteristics of the prospective service consumer, wherein the assigning includes calculating a probability of the prospective service consumer arriving at a service time window based on the historical arrival rate of the selected group. The assigning further includes calculating a reward for a reinforcement algorithm based on a selection criteria, wherein the selection criteria comprises the compatibility success score. Moreover, the assigning includes imposing a penalty function to the reinforcement algorithm for using the chosen service provider, wherein the penalty function is calculated based on unavailability of the chosen service provider.

In one or more embodiments, the performance indicator includes at least one of service cost, service time, service quality, service satisfaction, or service compliance. In one or more embodiments, the reward is a reference parameter. In one or more embodiments, the selection criteria further includes preferences of the service consumers.

One of ordinary skilled in the art would recognize that the methodology described in the above example is programmed into a software architecture which is differentiated by various protocols, wherein each discretized protocol is configured to execute a different method.

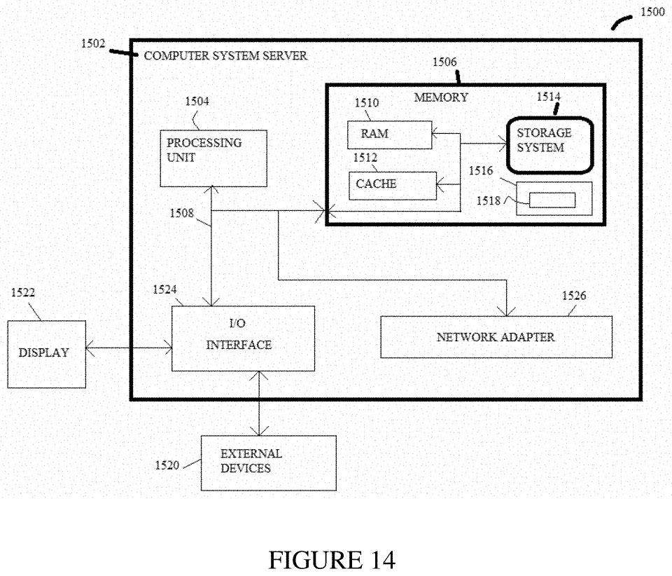

FIG. 14 illustrates one example of a computing or processing node 1500 for operating the methods and the software architecture of the present application. This is not intended to suggest any limitation as to the scope of use or functionality of embodiments of the invention described herein. Regardless, the computing node 1500 is capable of being implemented and/or performing any of the functionality set forth hereinabove.

In computing node 1500 there is a computer system/server 1502, which is operational with numerous other general purpose or special purpose computing system environments or configurations. Examples of well-known computing systems, environments, and/or configurations that may be suitable for use with computer system/server 1502 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 1502 may be described in the general context of computer system executable instructions, such as program modules, being executed by a computer system. Generally, program modules may include routines, programs, objects, components, logic, data structures, and so on that perform particular tasks or implement particular abstract data types. Computer system/server 502 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 14, computer system/server 1502 in cloud computing node 1500 is shown in the form of a general-purpose computing device. The components of computer system/server 1502 may include, but are not limited to, one or more processors or processing units 1504, a system memory 1506, and a bus 1508 that couples various system components including system memory 1506 to processor 1504.

Bus 1508 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 1502 typically includes a variety of computer system readable media. Such media may be any available media that is accessible by computer system/server 1502, and it includes both volatile and non-volatile media, removable and non-removable media.

System memory 1506, in one embodiment, implements the methods and the software architectures of the present application. The system memory 506 can include computer system readable media in the form of volatile memory, such as random access memory (RAM) 1510 and/or cache memory 1512. Computer system/server 1502 may further include other removable/non-removable, volatile/non-volatile computer system storage media. By way of example only, storage system 1514 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 1508 by one or more data media interfaces. As will be further depicted and described below, memory 1506 may include at least one program product having a set (e.g., at least one) of program modules that are configured to carry out the functions of various embodiments of the invention.

Program/utility 1516, having a set (at least one) of program modules 1518, may be stored in memory 1506 by way of example, and not limitation, as well as an operating system, one or more application programs, other program modules, and program data. Each of the operating system, one or more application programs, other program modules, and program data or some combination thereof, may include an implementation of a networking environment. Program modules 1518 generally carry out the functions and/or methodologies of various embodiments of the invention as described herein.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Computer system/server 1502 may also communicate with one or more external devices 1520 such as a keyboard, a pointing device, a display 1522, etc.; one or more devices that enable a user to interact with computer system/server 1502; and/or any devices (e.g., network card, modem, etc.) that enable computer system/server 1502 to communicate with one or more other computing devices. Such communication can occur via I/O interfaces 1524. Still yet, computer system/server 1502 can communicate with one or more networks such as a local area network (LAN), a general wide area network (WAN), and/or a public network (e.g., the Internet) via network adapter 1526. As depicted, network adapter 1526 communicates with the other components of computer system/server 1502 via bus 1508. It should be understood that although not shown, other hardware and/or software components could be used in conjunction with computer system/server 1502. Examples, include, but are not limited to: microcode, device drivers, redundant processing units, external disk drive arrays, RAID systems, tape drives, and data archival storage systems, etc.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, design, machine, manufacture, and composition of matter, means, methods and steps described in the specification. As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

While several embodiments have been provided in the present disclosure, it should be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

1. A non-transitory computer-readable medium encoded with a computer-readable program, which, when executed by a processor, will cause a computer to execute a method of intelligent matching, wherein the method comprises:
receiving a first data from a plurality of service vendors, wherein the first data comprises performance metrics of service providers and characteristics of service consumers;
classifying, using cluster algorithms, the service consumers from the first data into a plurality of groups;
calculating a compatibility success score between each service provider and each group of the plurality of groups;
calculating a historical arrival rate for the each group of the plurality of groups; and
assigning a chosen service provider to a prospective service consumer based on a determined compatibility success score of the chosen service provider, wherein the determined compatibility success score is the compatibility success score between the chosen service provider and a selected group of the plurality of group, wherein the selected group comprises the characteristics of the prospective service consumer.

2. The method of claim 1, wherein the receiving the first data further comprises:
receiving a second data, wherein the second data comprises: at least one of characteristics of service providers or preferences of service consumers.

3. The method of claim 2, wherein the classifying, using the cluster algorithms, the service consumers from the first data into the plurality of groups comprises:
classifying, using the cluster algorithms, the service consumers from the first data and the second data into the plurality of groups.

4. The method of claim 1, wherein the calculating the compatibility success score between the each service provider and the each group of the plurality of groups comprises:
obtaining statistical properties of distribution of a performance indicator of the at least one service provider associated with an entrusted group, wherein the at least one service provider has previously provided service to the entrusted group; and
ranking the at least one service providers based on the obtained statistical properties of distribution of the performance indicator.

5. The method of claim 4, wherein the calculating the compatibility success score between the each service provider and the each group of the plurality of groups comprises:
wherein an under-represented service provider of the at least one service provider does not have adequate statistical representation of the performance indicator in the entrusted group,
assigning statistical values to the under-represented service provider based on standard missing data procedures.

6. The method of claim 4, wherein the performance indicator comprises at least one of service cost, service time, service quality, service satisfaction, or service compliance.

7. The method of claim 6, further comprising:
ranking the at least one service providers based on the obtained statistical properties of distribution of the performance indicator, wherein at least one of the performance indicators are weighted by a user.

8. The method of claim 6, wherein the assigning the chosen service provider to the prospective service consumer based on the determined compatibility success score of the chosen service provider, wherein the determined compatibility success score is the compatibility success score between the chosen service provider and the selected group of the plurality of group, wherein the selected group comprises the characteristics of the prospective service consumer, comprises:
calculating a probability of the prospective service consumer arriving at a service time window based on the historical arrival rate of the selected group;
calculating a reward for a reinforcement algorithm based on a selection criteria, wherein the selection criteria comprises the compatibility success score; and
imposing a penalty function to the reinforcement algorithm for using the chosen service provider, wherein the penalty function is calculated based on unavailability of the chosen service provider.

9. The method of claim 8, wherein the reward is a reference parameter.

10. The method of claim 8, wherein the selection criteria further includes preferences of the service consumers.

11. The method of claim 8, wherein the calculating the reward for the reinforcement algorithm based on the selection criteria, wherein the selection criteria comprises the compatibility success score, comprises:
calibrating the reward based on external penalty functions from external reinforcement algorithm, wherein the external reinforcement algorithm and the reinforcement algorithm are part of a homogenous system implementation.

12. A non-transitory computer-readable medium encoded with a computer-readable program, which, when executed by a processor, will cause a computer to execute a method of intelligent matching, wherein the method comprises:
receiving a first data from a plurality of service vendors, wherein the first data comprises performance metrics of service providers and characteristics of service consumers;
classifying, using cluster algorithms, the service consumers from the first data into a plurality of groups;
calculating a compatibility success score between each service provider and each group of the plurality of groups, wherein the calculating comprises:
obtaining statistical properties of distribution of a performance indicator of the at least one service provider associated with an entrusted group, wherein the at least one service provider has previously provided service to the entrusted group;
ranking the at least one service providers based on the obtained statistical properties of distribution of the performance indicator;
calculating a historical arrival rate for the each group of the plurality of groups; and
assigning a chosen service provider to a prospective service consumer based on a determined compatibility success score of the chosen service provider, wherein the determined compatibility success score is the compatibility success score between the chosen service provider and a selected group of the plurality of group, wherein the selected group comprises the characteristics of the prospective service consumer.

13. The method of claim 12, wherein the performance indicator comprises at least one of service cost, service time, service quality, service satisfaction, or service compliance.

14. The method of claim 13, wherein the assigning the chosen service provider to the prospective service consumer based on the determined compatibility success score of the chosen service provider, wherein the determined compatibility success score is the compatibility success score between the chosen service provider and the selected group of the plurality of group, wherein the selected group comprises the characteristics of the prospective service consumer, comprises:
calculating a probability of the prospective service consumer arriving at a service time window based on the historical arrival rate of the selected group;
calculating a reward for a reinforcement algorithm based on a selection criteria, wherein the selection criteria comprises the compatibility success score; and
imposing a penalty function to the reinforcement algorithm for using the chosen service provider, wherein the penalty function is calculated based on unavailability of the chosen service provider.

15. The method of claim 14, wherein the reward is a reference parameter.

16. The method of claim 14, wherein the selection criteria further includes preferences of the service consumers.

17. A non-transitory computer-readable medium encoded with a computer-readable program, which, when executed by a processor, will cause a computer to execute a method of intelligent matching, wherein the method comprises:
receiving a first data from a plurality of service vendors, wherein the first data comprises performance metrics of service providers and characteristics of service consumers;
classifying, using cluster algorithms, the service consumers from the first data into a plurality of groups;
calculating a compatibility success score between each service provider and each group of the plurality of groups, wherein the calculating comprises:
obtaining statistical properties of distribution of a performance indicator of the at least one service provider associated with an entrusted group, wherein the at least one service provider has previously provided service to the entrusted group; and
ranking the at least one service providers based on the obtained statistical properties of distribution of the performance indicator;
calculating a historical arrival rate for the each group of the plurality of groups; and
assigning a chosen service provider to a prospective service consumer based on a determined compatibility success score of the chosen service provider, wherein the determined compatibility success score is the compatibility success score between the chosen service provider and a selected group of the plurality of group, wherein the selected group comprises the characteristics of the prospective service consumer, wherein the assigning comprises:
calculating a probability of the prospective service consumer arriving at a service time window based on the historical arrival rate of the selected group;
calculating a reward for a reinforcement algorithm based on a selection criteria, wherein the selection criteria comprises the compatibility success score; and
imposing a penalty function to the reinforcement algorithm for using the chosen service provider, wherein the penalty function is calculated based on unavailability of the chosen service provider.

18. The method of claim 17, wherein the performance indicator comprises at least one of service cost, service time, service quality, service satisfaction, or service compliance.

19. The method of claim 17, wherein the reward is a reference parameter.

20. The method of claim 17, wherein the selection criteria further comprises preferences of the service consumers.
